(19) United States
(12) Patent Application Publication
(10) Pub. No.: US 2005/0089879 A1
(43) Pub. Date: Apr. 28, 2005

(54) Method for producing arachidonic acid in transgenic organisms

(76) Inventors: Ivo Feussner, Halle/Saale (DE); Ellen Hornung, Quedlinburg (DE); Christian Pernstich, Berlin (DE); Martina Korfei, Lahntal-Sarnau (DE); Helmut Kindl, Marburg (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/769,647

(22) Filed: Jan. 30, 2004

Related U.S. Application Data
(63) Continuation of application No. PCT/EP02/08555, filed on Jul. 31, 2002.

(30) Foreign Application Priority Data
Jul. 31, 2001 (DE) 101 37 374.0

(51) Int. Cl. C12Q 1/68; C07H 21/04; C12P 7/64; C12N 9/04; A01H 1/00; C12N 15/82; C12N 5/04

(52) U.S. Cl. 435/6; 435/69.1; 435/190; 435/134; 435/419; 435/468; 536/23.2; 800/281

(57) ABSTRACT

The invention relates to a method for the production of arachidonic acid in transgenic organisms, especially in transgenic plants and yeasts. The invention also relates to DNA sequences coding for a protein with enzymatic activity of a Δ5-desaturase from Phytophthera megasperma. The invention further relates to transgenic plants and plant cell and transgenic yeasts containing a nucleic acid molecule comprising a DNA sequence according to the present invention and having, on the basis thereof, an enhanced arachidonic acid synthesis in comparison with wild-type cells. The invention also relates to harvest products and propagating material of transgenic plants.

### RELATED APPLICATIONS

This application is a continuation of International Application No. PCT/EP02/08555, filed Jul. 31, 2002, which claims priority to German Patent Application No. 101 37 374.0, filed Jul. 31, 2001, the disclosures of which are incorporated by reference herein in their entireties.

### BACKGROUND OF THE INVENTION

1. Field of the Invention

The invention relates to a method for the production of arachidonic acid in transgenic organisms, especially in transgenic plants and yeasts. The invention also relates to DNA sequences, which code for a protein having the enzymatic activity of a Δ5-desaturase from Phytophthora megasperma. The invention further relates to transgenic plants and plant cells and transgenic yeasts, containing a nucleic acid molecule comprising a DNA sequence according to the present invention and having, on the basis thereof, an increased arachidonic acid synthesis in comparison with wild-type cells. The invention also relates to harvest products and propagating material of transgenic plants.

2. Description of the Related Art

Unsaturated fatty acids are essential components which are required for a normal cellular function. Unsaturated fatty acids fulfill many different functions; they, for example contribute to membrane fluidity, or serve as signal molecules. A particular class of fatty acids, the polyunsaturated fatty acids (PUFA), have attracted great interest as pharmaceutical and nutritechnical compounds. PUFA can be defined as fatty acids with a length of 18 or more carbon atoms containing two or more double bonds. These double bonds are introduced by fatty acid-specific desaturase enzymes.

PUFA can be classified into two groups, n-6 or n-3, depending on the position (n) of the double bond that is closest to the methyl terminus of the fatty acid. Thus, γ-linolenic acid (18:3Δ^{6, 9, 12}) is classified as 18:3, n-6 PUFA, whereas α-linolenic acid (18:3Δ^{9, 12, 15}) is a 18:3, n-3 PUFA. Many PUFAs are also essential fatty acids that have to be present in the food in order to enable normal development in mammals that are unable to synthesize the primary essential PUFA fatty acid linoleic acid (18:2, n-6). The C20-fatty acid arachidonic acid (20:4, n-6) has been shown to be important for the health of neonates, including brain and eye development, and has recently attracted much attention as well.

Arachidonic acid (5,8,11,14-eicosatetraenic acid), as well as linoleic acid and linolenic acid, are counted among the essential fatty acids. The enzymatic oxidation of arachidonic acid leads to a number of biochemically important compounds, such as the prostaglandins, thromboxanes, prostacyclins, leukotrienes and lipoxins, all of which are called eicosanoids.

C20-fatty acids such as 20:4, n-6, are synthesized in the food from 18:2, n-6 by successive desaturation at Δ6, subsequent elongation to C-20 and further desaturation at Δ5.

FIG. 1 shows an outline of the biosynthesis of PUFAs in animals. Primary n-6 and n-3 fatty acids (such as linoleic acid and α-linolenic acid) have to be provided in the food, since animals are unable to further desaturate oleic acid. The enzyme activities that are required in this synthesis pathway are shown here.

Whereas arachidonic acid is the main metabolite of the n-6 biosynthesis pathway in mammals, the main end products of the n-3 pathway are eicosapentaenic acid (20:5, n-3) and docosahexaenic acid (22:6, n-3).

Arachidonic acid is present in fairly large amounts in the liver and in the adrenal glands, among others. It is also synthesized in the filamentous fungus Mortierella alpina and in the red alga Porphyridium cruentrum. Eicosapentaenic acid is found in fish oil, and in other marine organisms. However, many of these sources are not easily available for human use. Since plant oils are currently the greatest source of PUFAs in human nutrition, the modification of fatty acid biosynthesis pathways by genetic manipulation for production of the desired PUFA in an oil seed plant may provide an economic source of these important fatty acids. In recent years, fatty acid desaturase genes from various organisms have been cloned (Napier et al., Curr. Opin. Plant Biol. (1999) 2:123-127).

However, the Δ5-desaturase enzymes described in the art are not specific for dihomo-γ-linolenic acid (DGLA; 20:3, n-6). The work that has been conducted so far using relevant genes or gene products shows that the Δ5-desaturase enzymes known in the art not only convert 20:3- but also 20:2-, 20:1-, and in some cases also C18-fatty acids (see for example Beaudoin et al., Proc. Natl. Acad. Sci. USA (2000) 97: 6421-6426; Cho et al., J. Biol. Chem. (1999) 274: 37335-37339; Knutzon et al., J. Biol. Chem. (1998) 273: 29360-29366; Saito et al., Eur. J. Biochem. (2000) 267: 1813-1818). From this, important disadvantages arise which should be avoided, if possible. Due to the lack of specificity of the enzymes known in the art, only fatty acid mixtures are always generated, but it is desired to avoid this. As a result, not only defined long-chain PUFAs are generated, but also by-products that are not known in the literature, or that are without value, such as picolinic acid (see Knutzon et al., Saito et al., vide supra). Such enzymes are thus not suited, for example, for application in the food industry.

An enzyme is desired which enables the specific production and accumulation of one or a few fatty acids in a transgenic organism.

### SUMMARY OF THE INVENTION

In some embodiments, an isolated DNA sequence which codes for a protein having the enzymatic activity of a Δ5-desaturase that has enzymatic activity that is specific solely for dihomo-γ-linolenic acid is provided. The DNA sequence can originate, for example, from Phytophthora megasperma. Further, the DNA sequence can be selected from, for example,
a) a DNA sequence comprising a nucleotide sequence that codes for the amino acid sequence identified in SEQ ID No. 2 or a fragment thereof,
b) a DNA sequence comprising the coding nucleotide sequence given in SEQ ID No. 1 or a fragment thereof,
c) a DNA sequence comprising a nucleotide sequence, or a fragment of said nucleotide sequence, that hybridizes to a complementary strand of the coding nucleotide sequence from a) or b),
d) a DNA sequence comprising a nucleotide sequence, or fragment of said nucleotide sequence, which is degenerate to a nucleotide sequence from c), or
e) a DNA sequence representing a derivative, an analogue or a fragment of a coding nucleotide sequence from a), b), c) or d).

Also provided is a recombinant nucleic acid molecule having the above-described DNA sequence operably linked to a regulatory sequence of a promoter which is active in a target cell, preferably a plant cell or a yeast cell. Optionally, the nucleic acid sequence may also be operably linked to a regulatory sequence which can serve as transcription, termination and/or polyadenylation signals in the target cell.

In additional embodiments, a recombinant protein having the enzymatic activity of a Δ5-desaturase specific for dihomo-γ-linolenic acid is provided. The recombinant protein can originate, for example, from Phytophthora megasperma.

In some embodiments, a method for generating a plant cell or a yeast cell having an increased content of arachidonic acid compared to a wild-type plant or a wild-type cell is provided, by producing a recombinant nucleic acid molecule comprising the following elements in 5′→3′ direction: 1) a regulatory sequence of a promoter which is active in a plant cell or a yeast cell; and 2) a nucleic acid sequence which codes for a protein having the enzymatic activity of a Δ5-desaturase that is specific for dihomo-γ-linolenic acid operatively linked thereto; and transferring this nucleic acid molecule to a plant cell or a yeast cell. The nucleic acid molecule can also have at least one operably linked regulatory sequence which can serve as transcription, termination, and/or polyadenylation signals. In some embodiments, at least one transformed plant cell or at least one transformed yeast cell can be obtained. Additionally, a plant may be regenerated from the plant cell.

In some embodiments, a transgenic plant cell or a transgenic yeast cell having the isolated DNA sequence or recombinant nucleic acid which codes for a protein having the enzymatic activity of a Δ5-desaturase that has enzymatic activity that is specific solely for dihomo-γ-linolenic acid is provided. A transgenic plant produced according to any of the above methods is also provided.

In some embodiments, a transgenic plant or a plant part having the above-described nucleic acid molecule is provided. The transgenic plant or plant part can be, for example, a transgenic harvest product and a transgenic propagating material. The transgenic propagating material can be, for example, a protoplast, a plant cell, calli, a seed, a tuber, a cutting, or the transgenic progeny of the transgenic plant.

In some embodiments, a method for producing arachidonic acid in a transgenic plant, a plant cell, a yeast cell, or a cell culture, is provided, by transferring a nucleic acid molecule as described above to a plant, a plant cell, a yeast cell, or a cell culture, producing arachidonic acid by expression of the nucleic acid molecule in the transgenic cell, and obtaining arachidonic acid from the transgenic plant, the plant cell, the yeast cell, or the cell culture. The producing of arachidonic acid by expression of the nucleic acid molecule can comprise, for example, the addition of dihomo-γ-linolenic acid.

### BRIEF DESCRIPTION OF THE DRAWINGS

FIG. 1 shows an outline of PUFA biosynthesis in animals.

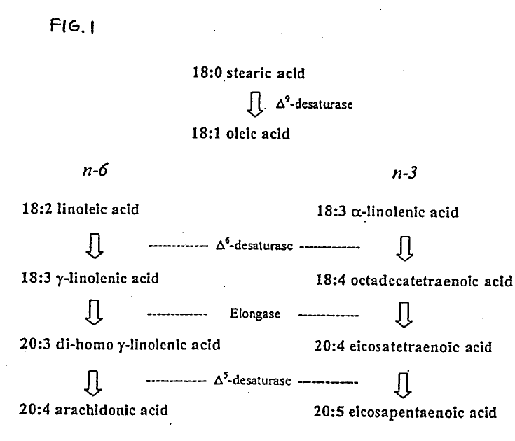

FIG. 2 shows the result of the GC analysis: FIG. 2a) yeast strain INVSc1 with pYES2 (control) and feeding with dihomo-γ-linolenic acid (dihomo-γ-LEA); FIG. 2b) yeast strain INVSc1 with pYES2+Δ5-desaturase and feeding with dihomo-γ-linolenic acid; FIG. 2c) standard.

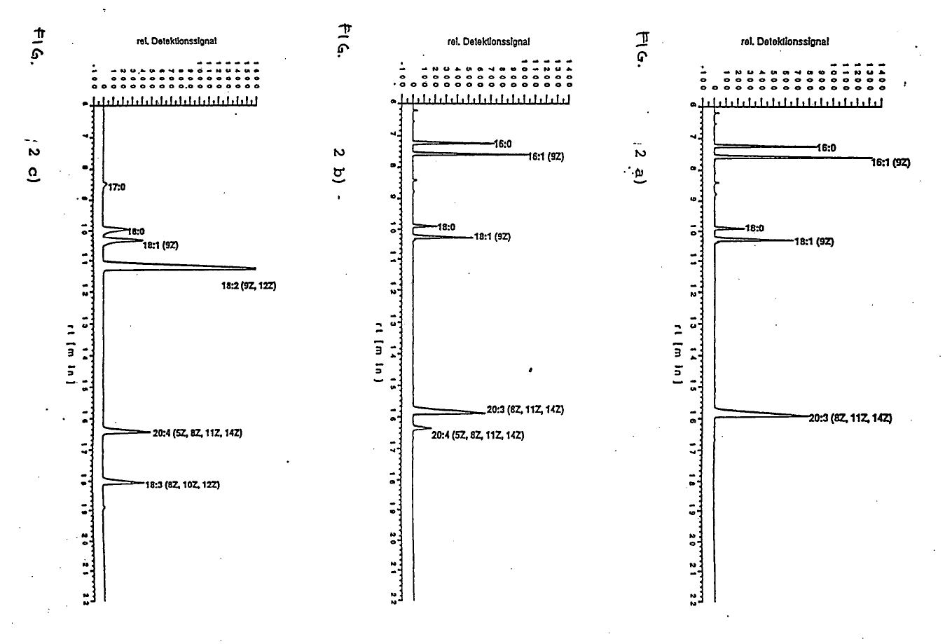

FIG. 3 shows the result of a GC analysis: Top: Yeast strain INVSc1 with pYES2 (control) and feeding with dihomo-γ-linolenic acid (dihomo-γ-LEA); Middle: Yeast strain INVSc1 with pYES2+Δ5-desaturase and feeding with dihomo-γ-linolenic acid; Bottom: Standard.

FIG. 4 shows the result of a GC analysis of a co-feeding experiment with three different C20-fatty acids: Top: Yeast strain INVSc1 with pYES2 (control) and feeding with dihomo-γ-linolenic acid (dihomo-γ-LEA, 20:3Δ^{8, 11, 14}), 20:2Δ^{13, 16} and 20:3Δ^{11, 14, 17}; Middle: Yeast strain INVSc1 with pYES2+Δ5-desaturase and feeding with dihomo-γ-linolenic acid; Bottom: Standard. Only arachidonic acid is detectable as a product.

### DETAILED DESCRIPTION OF THE PREFERRED EMBODIMENT

Now, a gene for an as yet unknown protein having the enzymatic activity of a Δ5-desaturase has been successfully isolated from the fungus Phytophthora megasperma. In contrast to the enzymes with Δ5-desaturase activity described in the art, this enzyme is specific for dihomo-γ-linolenic acid. For this reason, the gene described herein may be used especially advantageously for the expression of the Δ5-desaturase and thus for the enzyme-catalyzed production of arachidonic acid in transgenic plants and yeast cells.

Regarding the substrate specificity, the enzymes according to the invention are clearly different from the Δ5-desaturases described in the art. Whereas the known enzymes also convert other fatty acids besides 20:3 fatty acids, the enzymes of the invention solely convert 20:3. This high substrate specificity was completely unexpected in view of the specificities of other enzymes known in the art. Using simple substrate specificity analyses, e.g. expression in yeast, the enzymes of the invention can be distinguished from the enzymes known in the art.

The present invention thus relates to DNA sequences that code for a protein having the enzymatic activity of a Δ5-desaturase, with the activity being specific for dihomo-γ-linolenic acid.

By providing an enzyme having substrate specificity for 20:3 fatty acids, particularly dihomo-γ-linolenic acid, the disadvantages in the art are overcome, and 20:4 fatty acids, particularly arachidonic acid, are specifically produced in transgenic organisms.

Within the scope of the invention, a dihomo-γ-linolenic acid-specific activity means that the proteins having the enzymatic activity of a Δ5-desaturase and encoded by the DNA sequences according to the invention are essentially exclusively specific for dihomo-γ-linolenic acid (20:3, DHLA) and essentially do not convert other fatty acids. Particularly preferably, specificity for DHLA means that the Δ5-desaturase exclusively converts DHLA as a substrate. In other words, DHLA-specificity in the sense of the invention also means that the enzyme does not convert 20:2-, 20:1-, or C18-fatty acids in appreciable amounts, and preferably not at all.

In contrast to this, the Δ5-desaturases from Mortierella alpina and Dictyostelium discoideum described in the art also convert other fatty acids, which is associated with the disadvantages described above.

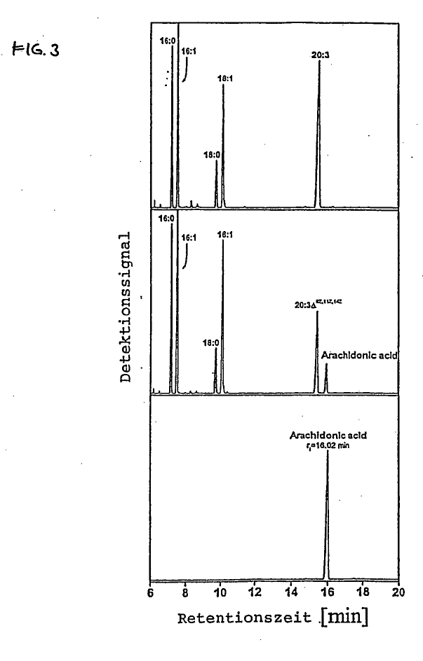

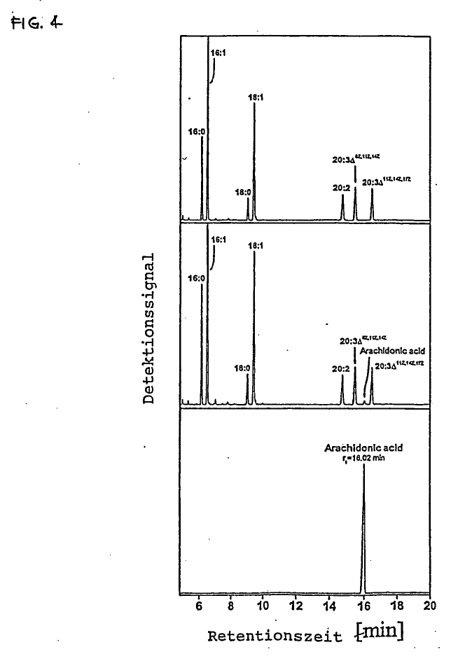

An expert can easily determine if a Δ5-desaturase has the DHLA-specificity according to the invention, and if the enzyme is thus a Δ5-desaturase in the sense of the present invention. For example, an expert can recognize from simple standard feeding experiments if an enzyme possesses the high substrate specificity for DHLA (see, for example, FIGS. 3 and 4). If the DHLA-specificity according to the invention is present, only DHLA is significantly converted to arachidonic acid, when, for example, various C20-fatty acids such as 20:2Δ^{11, 14}, 20:3Δ^{8, 11, 14} (DHLA) and 20:3Δ^{11, 14, 17} are offered as substrates in a feeding experiment.

Furthermore, the invention relates to recombinant nucleic acid molecules, comprising the following elements:
a) regulatory sequences of a promoter that is active in the target organism;
b) operatively linked thereto a DNA sequence that codes for a protein having the enzymatic activity of a Δ5-desaturase that is specific for dihomo-γ-linolenic acid;
c) optionally, operatively linked thereto regulatory sequences, which can serve as transcription, termination and/or polyadenylation signals in the target organism.

Preferably, the coding DNA sequence is the sequence from Phytophthora megasperma given in SEQ ID NO. 1. The target organism is preferably a plant or a plant cell, or a yeast cell, but other organisms such as fungi, algae may be considered as well.

The nucleic acid sequence given in SEQ ID NO. 1 shows sequence homologies to Δ5-desaturases from Dictyostelium discoideum (58%) and from Mortierella alpina (54%) as well as to Δ6-desaturases from Synchocystis sp. (46%) and Homo sapiens (43%), which are known in the art; and from which sequence homologies an expert would have been unable to distinguish between Δ5- or Δ6-acyl-lipid- or even Δ8-sphingolipid-desaturases (the determination of the sequence similarity was performed using the alignment program HUSAR BLAST X2).

The DNA sequence that codes for a protein having the enzymatic activity of a dihomo-γ-linolenic acid-specific Δ5-desaturase may be isolated from natural sources or may be synthesized according to conventional procedures. Using common molecular biological techniques (see for example Sambrook et al. (1989) Molecular Cloning: A Laboratory Manual, 2^{nd} ed., Cold Spring Harbor Laboratory Press, Cold Spring Harbor, N.Y.) it is possible to prepare or generate desired constructs for the transformation of plant cells or yeast cells. The methods of cloning, mutagenesis, sequence analysis, restriction analysis, and further biochemical or molecular biological methods commonly used for gene technological manipulation of prokaryotic cells are well known to an average person skilled in the art. Thus, not only can suitable chimeric gene constructs containing the desired fusion of promoter and Δ5-desaturase DNA sequence according to the invention, and, optionally, further regulatory and/or signal sequences, be generated; but also, the person skilled in the art can, if desired, additionally can introduce various mutations into the DNA sequence which codes for the Δ5-desaturase according to the invention using routine techniques, resulting in the synthesis of proteins with possibly altered biological properties. For example, it is possible to generate deletion mutants which permit the synthesis of correspondingly shortened proteins by progressive deletion from the 5′- or the 3′-end of the coding DNA sequence. Furthermore, it is possible to specifically produce enzymes that, by addition of suitable signal sequences, are localized in certain compartments of the plant cell or yeast cell. Such sequences are described in the literature and are well known to the person skilled in the art. Furthermore, the introduction of point mutations at positions in which a modification of the amino acid sequence affects for example the enzymatic activity or the regulation of the enzyme may be considered. In this way, mutants may be generated, for example, which are not subject anymore to the regulatory mechanisms that are usually present in the cell, such as allosteric regulation or covalent modification. Furthermore, mutants may be generated which show an altered substrate or product specificity. In addition, mutants may be generated which have an altered profile of activity, temperature, and/or pH.

For the gene technological manipulation in prokaryotic cells, the recombinant nucleic acid molecules according to the invention or parts thereof, may be introduced into plasmids that allow for mutagenesis or a sequence alteration by recombination of DNA sequences. Using standard methods (see e.g. Sambrook et al. (1989), supra), bases may be exchanged or natural or synthetic sequences may be added. In order to link the DNA fragments with each other, adapters or linkers may be attached to the fragments where necessary. In addition, suitable restriction sites may be provided, or unnecessary DNA or restriction sites may be removed using enzymatic or other manipulations. Where insertions, deletions or substitutions may be possible, in vitro mutagenesis, “primer repair”, restriction or ligation may be used. Sequence analysis, restriction analysis, and other biochemical-molecular biological methods are generally carried out as analytical methods.

In a preferred embodiment, the DNA sequence, which codes for a protein having the enzymatic activity of a Δ5-desaturase that is specific for dihomo-γ-linolenic acid is selected from the group consisting of a) DNA sequences, comprising a nucleotide sequence that codes for the amino acid sequence identified in SEQ ID No. 2 or fragments thereof, the length of the fragments being sufficient to be enzymatically active;
b) DNA sequences comprising the coding nucleotide sequence given in SEQ ID No. 1 or fragments thereof, the length of the fragments being sufficient to code for an enzymatically active protein;
c) DNA sequences comprising a nucleotide sequence or fragments of said nucleotide sequence, that hybridize to a complementary strand of the nucleotide sequence from a) or b), the length of the fragments being sufficient to code for an enzymatically active protein;
d) DNA sequences comprising a nucleotide sequence or fragments of said nucleotide sequence, which is degenerate to a nucleotide sequence from c), the length of the fragments being sufficient to code for an enzymatically active protein;
e) DNA sequences representing a derivative, analogue, or fragment of a nucleotide sequence from a), b), c) or d), the length of the fragment being sufficient to code for an enzymatically active protein.

Within the context of this invention, the term “hybridization” means hybridization under conventional hybridization conditions, preferably under stringent conditions, such as the ones described for example in Sambrook et al. (1989, vide supra).

According to the invention, hybridization is always performed in vitro under conditions that are stringent enough to ensure specific hybridization. Such stringent hybridization conditions are known to the expert, and may be derived from the literature (Sambrook et al. (2001), Molecular cloning: A laboratory manual, 3rd edition, Cold Spring Harbor Laboratory Press).

Generally, “to hybridize specifically” means that a molecule preferentially binds to a specific nucleotide sequence under stringent conditions, when this sequence is present in a complex mixture of (e.g. total) DNA or RNA. The term “stringent conditions” generally stands for conditions, under which a nucleic acid sequence preferentially hybridizes to its target sequence, and to a noticeably lesser extent, or not at all, to other sequences. Stringent conditions are partly sequence-dependent, and will vary depending on the different circumstances. Longer sequences hybridize specifically at higher temperatures. Generally, stringent conditions are selected in such a way that the temperature is approx. 5° C. below the thermal melting point (Tₘ) for the specific sequence at a defined ionic strength and a defined pH. The Tₘ represents the temperature (at a defined ionic strength, pH and nucleic acid concentration) at which 50% of the molecules complementary to the target sequence hybridize to the target sequence in the state of equilibrium. Typically, stringent conditions are conditions at which the salt concentration is at least about 0.01 to 1.0 M sodium ion concentration (or another salt) at a pH between 7.0 and 8.3, and the temperature is at least about 30° C. for short molecules (i.e. for example 10-50 nucleotides). Additionally, stringent conditions, as already described above, may be obtained by addition of destabilizing agents such as for example formamide.

DNA sequences that hybridize to DNA sequences coding for a protein having the enzymatic activity of a Δ5-desaturase with the specificity for dihomo-γ-linolenic acid, may be isolated e.g. from genomic or cDNA libraries of any organism that naturally contains the Δ5-desaturase coding DNA sequences of the invention. The identification and isolation of such DNA sequences may be conducted e.g. using DNA sequences having exactly or essentially the nucleotide sequence given in SEQ ID No. 1 or fragments thereof, or the reversely complementary sequences of these DNA sequences, e.g. by hybridization according to standard procedures (see, e.g. Sambrook et al. (1989), vide supra). The fragments used as hybridization probes may also be synthetic fragments that have been generated using common synthesis techniques, the sequence of which essentially corresponds to one of the above mentioned Δ5-desaturase DNA sequences, or to a fragment thereof. DNA sequences according to the invention may of course be isolated using other procedures such as e.g. PCR as well.

The DNA sequences which code for a protein having the biological activity of a Δ5-desaturase with specificity for dihomo-γ-linolenic acid also comprise DNA sequences, the nucleotide sequences of which are degenerate to any of the previously described DNA sequences. Degeneracy of the genetic code offers the person skilled in the art, among other things, the possibility of adapting the nucleotide sequence of the DNA sequence to the codon preference (codon usage) of the target organism, i.e. the plant or plant cell or yeast cell having an altered content of arachidonic acid as a result of the expression of the enzymatic activity according to the invention, and thereby optimizing the expression.

The above described DNA sequences also comprise fragments, derivatives, and allelic variants of the above described DNA sequences which code for a protein having the biological activity of a Δ5-desaturase specific for dihomo-γ-linolenic acid. The term “fragments” is to be understood as parts of the DNA sequence that are long enough to code for one of the described proteins. The term “derivative” in this context means that the sequences differ from the above described DNA sequences in one or more positions, but still possess a high degree of homology to these sequences.

In this respect, homology means a sequence identity of at least 60 percent, 64 percent, 68 percent, especially an identity of at least 70 percent, 72 percent, 74 percent, 76 percent, 78 percent, preferably of at least 80 percent, 82 percent, 84 percent, 86 percent, 88 percent, particularly preferred of at least 90 percent, 92 percent, 94 percent, and most preferably of at least 95 percent, 97 percent, 99 percent.

The enzymes that are coded by these DNA sequences show a sequence identity to the amino acid sequence given in SEQ ID No. 2 of at least 60, 64, 68, 70, 74, 78 percent, especially of at least 80, 82 percent, preferably of at least 84, 86, 88, 90 percent, and particularly preferred of at least 92, 94, 96, 98 percent. The deviations from the above described DNA sequences may have occurred for example due to deletion, substitution, insertion or recombination.

Degrees of homology or sequence identities are generally determined using various alignment programs such as e.g. CLUSTAL. Generally, algorithms that are suitable for the determination of sequence identity/similarity are available to the expert, such as the program, which is accessible under hypertext transfer protocol (http) on the world wide web at ncbi.nlm.nih.gov/BLAST (e.g. the link “Standard nucleotide-nucleotide BLAST [blastn]”).

The DNA sequences that are homologous to the above described sequences and are derivatives of these sequences are normally variations of these sequences which represent modifications fulfilling the same biological function. These variations can be both naturally occurring variations, for example sequences from other organisms, or mutations, whereby these mutations can have been generated naturally, or may have been introduced by targeted mutagenesis. Furthermore, the variations can be synthetic sequences. The allelic variants may be both naturally occurring variants and synthetic variants or variants generated by recombinant DNA techniques.

In an especially preferred embodiment, the described DNA sequence which codes for a Δ5-desaturase that is specific for dihomo-γ-linolenic acid is obtained from Phytophthora megasperma.

Furthermore, the invention relates to nucleic acid molecules that contain the nucleic acid sequences according to the invention, or that have been generated or derived from these by naturally occurring processes or by gene technological or chemical processes and synthesis methods. These may be, for example, DNA or RNA molecules, cDNA, genomic DNA, mRNA, etc.

The invention also relates to those nucleic acid molecules in which the nucleic acid sequences are linked to regulatory elements, which ensure the transcription and, if desired, the translation in the transgenic cell.

For the expression of the DNA sequences contained in the recombinant nucleic acid molecules according to the invention in plant cells, basically any promoter can be used that is active in plant cells. Thus, the DNA sequences according to the invention may be expressed in plant cells for example under the control of constitutive, but also inducible or tissue- or development-specific regulatory elements, especially promoters. Whereas for example the use of an inducible promoter allows for the targeted induced expression of the DNA sequences according to the invention in plant cells, the use of tissue-specific, e.g. leaf- or seed-specific, promoters for example offers the possibility of altering the content of arachidonic acid in specific tissues, such as leaf or seed tissues. Other suitable promoters mediate for example light-induced gene expression in transgenic plants. With respect to the plant to be transformed, the promoter may be homologous or heterologous.

Suitable promoters are for example the 35S RNA promoter of the cauliflower mosaic virus and the ubiquitin promoter from maize for constitutive expression. Suitable seed specific promoters are, for instance, the USP promoter (Bäumlein et al., Mol. Gen. Genet. (1991) 225: 459-467) or the hordein promoter (Brandt et al., Carlsberg Res. Commun. (1985) 50: 333-345).

Within the scope of this invention, constitutive, germination-specific and seed-specific promoters are preferred, since they are particularly suitable for the targeted increase of the arachidonic acid content in transgenic seeds.

Anyway, the expert can gather suitable promoters from the literature, or can isolate them himself from any plant using routine methods.

This also applies to the expression of the DNA sequences according to the invention in yeast cells. Inducible promoters, or for example the OLE 1 promoter may be preferably used in this case.

Furthermore, transcription or termination sequences are present which allow for correct transcription termination, as well as for the addition of a poly-A tail to the transcript to which a function in the stabilization of transcripts is assigned. Such elements are described in the literature (e.g. Gielen, EMBO J., (1989) 8:23-29), and are interchangeable in any order, for example the terminator of the octopine synthase gene from Agrobacterium tumefaciens.

The invention further relates to proteins having the biological activity of a Δ5-desaturase that is specific for dihomo-γ-linolenic acid, or biologically active fragments thereof, which are coded by a nucleic acid sequence according to the invention or by a nucleic acid molecule according to the invention. Preferably, it is a fungal Δ5-desaturase, particularly preferably it is a protein having the amino acid sequence shown in SEQ ID NO. 2, or an active fragment thereof.

A further object of the invention is to provide vectors, the use of which enables the production of new plants, or plant cell or tissue cultures, or yeasts in which an altered content of arachidonic acid can be achieved. This object is solved by providing the vectors according to the invention containing nucleic acid sequences, which code for enzymes having the biological activity of a Δ5-desaturase according to the invention.

Thus, the present invention also relates to vectors, especially plasmids, cosmids, viruses, bacteriophages, and other vectors that are common in gene technology containing the previously described nucleic acid molecules according to the invention, and which may be optionally used for the transfer of the nucleic acid molecules according to the invention to plants or to plant cells or yeast cells.

Optionally, the nucleic acid sequences of the invention can further contain enhancer sequences or other regulatory sequences. These regulatory sequences also include, for example, signal sequences, which enable the transport of the gene product to a specific compartment.

It is also an object of the invention to provide new transgenic plants, plant cells, plant parts, transgenic propagation material and transgenic harvest products having an altered content of arachidonic acid compared to wild-type plants or plant cells.

This object is solved by transfer of the nucleic acid molecules according to the invention to plants and their expression in plants. By providing the nucleic acid molecules according to the invention it is now possible to alter plant cells by gene technological procedures, so that they have a novel or altered Δ5-desaturase activity with the specificity for dihomo-γ-linolenic acid compared to wild-type cells, thus resulting in an alteration of the content of arachidonic acid.

In one embodiment the invention thus relates to plants or their cells and parts, having an increased content of arachidonic acid compared to wild-type plants due to the presence and expression of the nucleic acid molecules according to the invention.

Further subjects of the invention are transgenic plant cells, or plants comprising such plant cells and their parts and products, in which the nucleic acid molecules according to the invention are integrated into the plant genome. Further subjects of the invention are plants, in whose cells the nucleic acid sequence according to the invention is present in replicated form, i.e. the plant cell contains the foreign DNA on a separate nucleic acid molecule (transient expression).

Plants which are transformed with the nucleic acid molecules according to the invention and in which an altered amount of arachidonic acid is synthesized due to the introduction of such a molecule, can in principle be any plant. Preferably the plant is a monocotyledonous or a dicotyledonous useful plant.

Examples for monocotyledonous plants are plants belonging to the genera Avena (oats), Triticum (wheat), Secale (rye), Hordeum (barley), Oryza (rice), Panicum, Pennisetum, Setaria, Sorghum (millet), Zea (maize). Dicotyledonous useful plants are, among others, leguminous plants, and especially alfalfa, soy bean, rape, tomato, sugar beet, potato, ornamental plants or trees. Other useful plants may be e.g. fruits (particularly apples, pears, cherries, grapes, citrus fruits, pineapple and banana), oil palms, tea shrubs, cacao and coffee trees, tobacco, sisal, cotton, flax, sunflower as well as medicinal plants and pasture grasses as well as fodder plants. Particularly preferred are wheat, rye, oat, barley, rice, maize and millet, fodder corns, sugar beet, rape, soy, tomato, potato, sweet grass, fodder grass, and clover.

It goes without saying that the invention especially relates to common food or fodder plants, such as, in addition to the already mentioned plants, peanut, lentil, field bean, mangel-wurzel, buckwheat, carrot, sunflower, Jerusalem artichoke, turnip rape, white mustard, rutabaga and wild turnip.

Especially preferred are oil seeds, and here especially the so-called 18:2-plants, i.e. soy bean, flax and sunflower.

Subject of the invention are further propagation material and harvest products of plants according to the invention, such as seeds, fruits, cuttings, bulbs, rhizomes, etc., as well as parts of these plants, such as protoplasts, plant cells and calli.

However, the invention not only relates to the increase of the arachidonic acid content in plants with the purpose of increasing the nutritional value for the nutrition of humans and animals. Rather, the invention also relates to the production of arachidonic acid in cell cultures with the purpose of subsequently obtaining arachidonic acid from the transgenic cells. This may be plant cell cultures such as suspension cultures or callus cultures, but also yeast cultures that are suitable for the production of arachidonic acid and subsequent isolation of arachidonic acid. Of course, transgenic plants or parts from transgenic plants may also be used for the production of arachidonic acid in order to obtain the arachidonic acid from these plants or plant parts. As with plants or plant cultures, yeasts or yeast cultures may not only be used for isolation of the arachidonic acid from the transgenic, i.e. arachidonic acid-producing cells, but also they may be directly suitable as foods with increased nutritional value.

If required, the transgenic, arachidonic acid-producing cells may be supplemented with dihomo-γ-linolenic acid to provide sufficient starting material for the synthesis of arachidonic acid.

For obtaining the arachidonic acid from the transgenic cells, plants or cell cultures, conventional procedures are suitable, such as e.g. cold pressing, which is also used for olive oil. In order to obtain arachidonic acid from yeast cells or yeast cultures, steam distillation may be suitable as well. The expert can gather suitable methods from the art.

For the cultivation of transgenic plant cells, common cell culture methods may be used which are known to the expert. The same applies to the cultivation of yeast cells.

In a further embodiment, the invention relates to host cells, especially prokaryotic and eukaryotic cells, that have been transformed or infected with an above described nucleic acid molecule or a vector, respectively, as well as cells, that are derived from such host cells, and that contain the described nucleic acid molecules or vectors. The host cells may be bacteria, viruses, algae, yeast cells and fungal cells, as well as plant or animal cells.

It is also an object of the present invention to provide methods for the production of plant cells and plants characterized by an increased content of arachidonic acid.

This object is solved by methods rendering possible the production of new plant cells and plants having an increased content of arachidonic acid due to the transfer of nucleic acid molecules according to the invention that code for Δ5-desaturase that is specific for dihomo-γ-linolenic acid. For the production of such new plant cells and plants, various methods may be used. Firstly, plants and plant cells may be modified by common gene technological transformation methods in such a way that the new nucleic acid molecules are integrated into the plant genome, i.e. stable transformants are generated. Secondly, a nucleic acid molecule according to the invention, the presence and the expression of which results in an altered biosynthesis performance in the plant cell, may be present in the plant cell or in the plant as a self-replicating system. Thus, the nucleic acid molecules of the invention may be present e.g. in a virus, with which the plant or plant cells comes in contact.

According to the invention, plant cells and plants having an increased content of arachidonic acid due to the expression of a nucleic acid sequence according to the invention, are produced by a method comprising the following steps:
a) Producing a recombinant nucleic acid molecule, comprising the following elements in 5′→3′ orientation:
   regulatory sequences of a promoter which is active in plant cells,
   operatively linked thereto a nucleic acid sequence which codes for a protein having the enzymatic activity of a Δ5-desaturase that is specific for dihomo-γ-linolenic acid, and
   optionally, operatively linked thereto regulatory sequences, which can serve as transcription, termination and/or polyadenylation signals in plant cells.
b) transferring the nucleic acid molecule from a) to plant cells.

In the case of yeast cells, the above-described method is modified in such way that regulatory sequences of a promoter active in yeast cells and the corresponding transcription, termination and/or polyadenylation signals are used.

In order to prepare the introduction of foreign genes into higher plants or their cells, a large number of cloning vectors are available which contain a replication signal for E. coli and a marker gene for the selection of transformed bacterial cells. Examples of such vectors are pBR322, the pUC series, the M13 mp series, pACYC184, etc. The desired sequence may be introduced into the vector at a suitable restriction site. The obtained plasmid is then used for the transformation of E. coli cells. Transformed E. coli cells are grown in a suitable medium, and then harvested and lysed in order to recover the plasmid. Restriction analyses, gel electrophoresis, and other biochemical-molecular biological methods are generally used as analytic methods to characterize the plasmid DNA so obtained. After each manipulation, the plasmid DNA may be cleaved and the DNA fragments thus obtained can be linked to other DNA sequences.

A plurality of techniques is available for introducing DNA into a plant host cell, and the person skilled in the art will not have any difficulties in selecting a suitable method in each case. These techniques comprise the transformation of plant cells with T-DNA using Agrobacterium tumefaciens or Agrobacterium rhizogenes as the transforming agent, the fusion of protoplasts, injection, electroporation or direct gene transfer of isolated DNA into protoplasts, introduction of DNA using biolistic methods, as well as other possibilities which have been well established for several years and which belong to the standard repertoire of the person skilled in the art in plant molecular biology or plant biotechnology and in cell and tissue cultures, and which are described in generally known review articles and manuals regarding the transformation of plants.

Once the introduced DNA has been integrated into the genome of the plant cell, it is generally stable there, and is maintained in the progeny of the originally transformed cell as well. It usually contains a selection marker which imparts the transformed plant cells resistance to a biocide or an antibiotic such as kanamycin, G 418, bleomycin, hygromycin, methotrexate, glyphosate, streptomycin, sulfonylurea, gentamycin or phosphinotricin, and others. The individually selected marker should therefore allow for the selection of transformed cells from cells lacking the introduced DNA. Alternative markers may also be suitable for this purpose, such as nutritive markers, screening markers (such as GFP, green fluorescent protein). It could also be done without any selection marker, although this would involve a rather high screening effort. If the selection marker used is to be removed after transformation and identification of successfully transformed cells and plants, the expert can choose among various strategies. For example, sequence-specific recombinases may be used, for example by performing retransformation of a recombinase-expressing parent line, followed by outcrossing of the recombinase after removal of the selection marker. The selection marker may also be removed by cotransformation followed by outcrossing.

If desired, transgenic plants are regenerated from transgenic plant cells by conventional regeneration methods using conventional culture media and phytohormones. If desired, the plants so obtained may be analyzed for the presence of the introduced DNA which codes for a protein having the enzymatic activity of a dihomo-γ-linolenic acid-specific Δ5-desaturase, or for the presence of enzyme activity attributed to the Δ5-desaturase using conventional methods including molecular biological methods, such as PCR, blot analyses, or by biochemical methods.

It is understood that plant cells containing the nucleic acid molecules according to the invention may also be further cultivated as plant cells (including protoplasts, calli, suspension cultures and the like). The invention also relates to the production of arachidonic acid in plant cultures.

According to the invention, the term transgenic plant comprises the plant as a whole, as well as all transgenic plant parts, in which a desaturase according to the invention is expressed. Such plant parts may for example be plant cells, plant seeds, leaves, blossoms, fruits, storage organs such as bulbs, and pollen. “Transgenic plant” according to the invention is also intended to mean the propagation material of transgenic plants according to the invention, such as e.g. seeds, fruits, cuttings, bulbs, rhizomes, etc., whereby this propagation material optionally contains the above described transgenic plant cells, as well as transgenic parts of these transgenic plants such as protoplasts, plant cells and calli.

The transformation, expression and cultivation of yeast cells may be accomplished according to conventional protocols, e.g. as described in Ausubel et al., Current Protocols in Molecular Biology (2000). Examples for this are also given in the embodiments below.

The invention furthermore relates to a method of obtaining arachidonic acid from plant cells or yeast cells. The literature provides the expert with protocols for the purification of C20-fatty acids and especially for the purification of arachidonic acid.

The following examples are intended to illustrate the invention without restricting it in any way.

### EXAMPLES

### Example 1

### Isolation of a cDNA Coding for a Δ5-desaturase from Phytophthora megasperma

Poly(A)⁺-RNA was isolated from 2 days old Phytophthora megasperma mycelia that had been cultivated on an oleate-containing medium (Oligotex-dT mRNA Maxi Kit, Qiagen, Hilden, Germany). cDNA synthesis was then performed using 5 μg of the poly(A)⁺-RNA and the Superscript™ cDNA synthesis kit from LifeTechnologies (Eggenstein, Germany). The obtained cDNA had asymmetrical ends. The cDNA was then ligated into the plasmid pSPORT-1 (LifeTechnologies, Eggenstein, Germany) that had already been cut with the restriction enzymes SalI and NotI. The ligation was performed using T4 ligase according to the manufacturer's protocol (LifeTechnologies).

Cells of the E. coli strain XL1-Blue were then transformed with the ligation mixture, i.e. the cDNA ligated into pSPORT-1 by electroporation, and plated on LB plates containing ampicillin, so that 10,000 to 15,000 colonies grew on each agar plate having a diameter of 15 cm.

The colonies were transferred from the agar plates to 2 nitrocellulose filters each, and then hybridization was conducted according to state of the art (Sambrook et al. 1989 supra) using α-[³²P]-dCTP labeled nucleotide sequences. Colonies that showed a hybridization signal were cultivated and sequenced.

The cDNA library was screened using the following nucleotide sequences:

(SEQ ID NO. 3)

Oligo 1
5′-GTG GCC AAG CAC AAC ACG GCC AAG AGC-3′

(SEQ ID NO. 4)

Oligo 2
5′-ACC ATC CGC GGC GTC GTC TAC GAC GTG ACC-3′

A positive cDNA clone is given in SEQ ID No. 1. This DNA sequence from P. megasperma codes for an enzyme having the activity of a Δ5-desaturase with 20:3-substrate specificity.

### Example 2

### Expression of the Δ5-Desaturase cDNA Clone in Yeast

First of all, the cDNA clone was cloned into the yeast expression vector pYES2 (Invitrogen, Groningen, Netherlands). This was carried out using the following oligonuclotide primers:

(SEQ ID NO. 5)

5′-GGA TCC ATG GCC CCC ATC GAG ACT GTC AAA G3′
(primer a)
and

(SEQ ID NO. 6)

5′-GCA TGC CCC GCG TTA TCC AGC CAA AGC TTA CC3′
(primer b)

Primer a contains a restriction site for BamHI (underlined), primer b contains a restriction site for the restriction enzyme SphIb (underlined).

The PCR was carried out according to the following PCR protocol:

Set up for PCR reaction:

| | | | |
|---|---|---|---|
| dNTP mix | 1 | μl | |
| 5′-primer (primer a) | 4 | μl | |
| 3′-primer (primer b) | 4 | μl | |
| template | 1 | μl | plasmid DNA (10 ng) |
| polymerase | 0.5 | μl | high fidelity, Roche Diagnostics GmbH |
| 10 × buffer | 5 | μl | |
| water | 34.5 | μl | |
| total volume | 50 | μl | |

The PCR was performed under the following conditions:
2 min 94° C.
10 cycles, each with: 30 seconds at 94° C., 30 seconds at 55° C., 1 minute at 72° C.
15 cycles with a time increment of 5 seconds: 30 seconds at 94° C., 30 seconds at 55° C., 1 minute at 72° C.
5 minutes at 72° C.

The obtained PCR fragment was then ligated into the vector pGEM-T (Promega, Madison, USA) for an intermediate cloning step. The ligation reaction was transformed into XL1-Blue cells.

The miniprep DNA obtained from the transformed cells (from 5 ml overnight culture, 37° C., processed with spin-prep kit from Macherey & Nagel, Düren, Germany) was digested with the restriction enzymes BamHI and PaeI from MBI Fermentas (St. Leon-Rot, Germany).

Then, using T4 ligase from MBI Fermentas, it was ligated into the yeast expression vector pYES2 that had been already cut also with the restriction enzymes BamHI and PaeI. The ligation reaction was transformed into E. coli-XL1-Blue cells.

5 ml of overnight cultures from various clones were analyzed for their plasmid DNA (miniprep DNA isolation with the spin prep kit from Macherey & Nagel). The desired clone (in sense-orientation behind the galactose promoter Gal1) was transformed into the yeast strain INVSc1 (Invitrogen, Groningen, Netherlands) using the lithium acetate method (Ausubel et al. (2000) supra).

The transformed yeast cells were grown overnight at 30° C. in SD medium (Ausubel et al. (2000) supra), containing glucose (2%) and amino acid solution, without uracil. After the pre-culture had been washed twice without sugar, a main culture was subsequently cultivated for 72 hours at 30° C. in SD medium, containing galactose and amino acid solution, without uracil, and containing linoleic acid (20 mg, 0.02%) or dihomo-γ-linolenic acid (15 mg, 0.015%) and Tergitol (10%).

This main culture was then harvested by centrifugation, lyophilization of the samples and transmethylation of the samples using sodium methylate.

### Example 3

### Expression in Yeast Cells

All yeast techniques are state of the art according to Ausubel et al., Current Protocols in Molecular Biology, 2000.

Pre-culture: 20 ml SD medium+glucose+amino acid solution without the respective amino acid for the selection were inoculated with a single colony, incubated overnight at 30° C. and shaken at 140 rpm.

Main culture: The pre-culture was washed twice (by centrifugation and resuspension) in SD medium without sugar, and the main culture is then inoculated to an OD₆₀₀ of 0.1-0.3. Cultivation of the main culture was performed in SD medium+galactose+amino acid solution without the respective amino acid+fatty acid, Tergitol NP40 (10%) at 30° C. for 72 h with shaking at 140 rpm. Harvesting of the main culture was performed by centrifugation in 50 ml sterile centrifuge tubes.

Lyophilization of the yeast cell pellet: Frozen cell pellets were lyophilized for approx. 18 h.

### Example 4

### Transmethylation

Dry sample+1.35 ml of methanol:toluene (2:1)+0.5 ml Na-methoxide solution—have to be ground very finely with a glass rod.

Incubate for 1 h at RT shaking on a Belly Dancer® Shaker.

Add 1.8 ml of 1M NaCl solution.

Add 2 full pasteur pipettes of n-heptane.

Extract for 10 min shaking on a Belly Dancer® Shaker.

Centrifuge (10 min, 400 rpm, 4° C.).

Transfer heptane supernatant into a test tube.

Evaporate solvents under N₂.

Transfer with 3×0.3 ml of hexane into Eppendorf tube.

Evaporate solvents under N₂, and resuspend residue in MeCN.

### Example 5

### GC Analysis

7 μl sample (in MeCN) in a sample tube.

Inject 1 μl into the GC.

5 μl sample was evaporated under nitrogen flow. The residue was taken up in 400 μl MeOH and 10 μl EDAC solution (10 ml EDAC/100 μl MeOH) were added.

Then, it was shaken for 2 hours at room temperature. After the addition of 200 μl tris buffer (0.1 M, pH 7.4), it was extracted twice by shaking, each with 1 ml hexane. The hexane phases were combined, and evaporated under nitrogen. The residue was dissolved in 20 μl MeCN.

The GC analysis was conducted under the following conditions:
Column: HP-INNOWax (cross-linked PEG), 30 m×0.32 mm×0.5 μm
Flow rate: 1.5 ml/min (constant flow) helium 150° C.
Injection: 220° C.
Oven: 150° C. (1 min), to 200° C. (15 K/min), to 250° C. (2 K/min), 250° C. (5 minutes)
Detection: FID 275° C.

In feeding experiments, it could be clearly confirmed that the desaturases according to the invention are highly specific for DGLA, i.e. these enzymes convert only this substrate detectably to arachidonic acid.

Within the scope of the feeding experiments, the yeast strain INVSc1 was usually cultivated for 3 days at 30° C. on medium containing different C20-fatty acids depending on the experiment. The yeast strain INVSc1 containing the control vector pYES2 without the desaturase sequence was used as a control.

1. An isolated DNA sequence which codes for a protein having the enzymatic activity of a Δ5-desaturase, wherein the enzymatic activity is specific solely for dihomo-γ-linolenic acid.

2. The DNA sequence according to claim 1, wherein the sequence is selected from the group consisting of:
a) a DNA sequence comprising a nucleotide sequence that codes for the amino acid sequence identified in SEQ ID No. 2 or a fragment thereof,
b) a DNA sequence comprising the coding nucleotide sequence given in SEQ ID No. 1 or a fragment thereof,
c) a DNA sequence comprising a nucleotide sequence, or a fragment of said nucleotide sequence, that hybridizes to a complementary strand of the coding nucleotide sequence from a) or b),
d) a DNA sequence comprising a nucleotide sequence, or fragment of said nucleotide sequence, which is degenerate to a nucleotide sequence from c),
e) a DNA sequence representing a derivative, an analogue or a fragment of a coding nucleotide sequence from a), b), c) or d).

3. The DNA sequence according to claim 1, originating from Phytophthora megasperma.

4. The DNA sequence according to claim 2; originating from Phytophthora megasperma.

5. A recombinant nucleic acid molecule, comprising:
a regulatory sequence of a promoter which is active in a target cell, preferably a plant cell or a yeast cell, operatively linked to a DNA sequence according to claim 1.

6. The recombinant nucleic acid molecule of claim 5, further comprising a regulatory sequence operatively linked thereto, which can serve as transcription, termination and/or polyadenylation signals in the target cell.

7. A recombinant protein having the enzymatic activity of a Δ5-desaturase, wherein the enzymatic activity is specific for dihomo-γ-linolenic acid.

8. The recombinant protein according to claim 7, originating from Phytophthora megasperma.

9. A method for generating a plant cell or a yeast cell having an increased content of arachidonic acid compared to a wild-type plant or a wild-type cell, comprising:
a) producing a recombinant nucleic acid molecule comprising the following elements in 5′→3′ direction:
a regulatory sequence of a promoter which is active in a plant cell or a yeast cell,
a nucleic acid sequence, which codes for a protein having the enzymatic activity of a Δ5-desaturase that is specific for dihomo-γ-linolenic acid, operatively linked thereto; and
b) transferring the nucleic acid molecule from a) to a plant cell or a yeast cell.

10. The method of claim 9, wherein the nucleic acid molecule further comprises at least one operably linked regulatory sequence which can serve as transcription, termination, and/or polyadenylation signals.

11. The method of claim 9, further comprising obtaining at least one transformed plant cell or at least one transformed yeast cell.

12. The method of claim 9, further comprising regenerating a plant from the plant cell.

13. A transgenic plant cell or a transgenic yeast cell comprising a DNA sequence or a recombinant nucleic acid molecule according to claim 1.

14. A transgenic plant cell or a transgenic yeast cell comprising a DNA sequence or a recombinant nucleic acid molecule according to claim 2.

15. A transgenic plant cell or a transgenic yeast cells comprising a DNA sequence or a recombinant nucleic acid molecule produced by a method according to claim 9.

16. A transgenic plant comprising a plant cell produced according to the method of claim 9.

17. A transgenic plant produced according to the method of claim 12.

18. A transgenic plant or a plant part, comprising the nucleic acid molecule according to claim 1, wherein said transgenic plant or plant part is selected from the group consisting of: a transgenic harvest product and a transgenic propagating material.

19. The transgenic plant or plant part of claim 18, wherein said transgenic propagating material is selected from the group consisting of: a protoplast, a plant cell, calli, a seed, a tuber, a cutting, and the transgenic progeny of the transgenic plant.

20. A method for producing arachidonic acid in a transgenic plant, a plant cell, a yeast cell, or a cell culture, comprising:
a) transferring a nucleic acid molecule according to claim 1 to a plant, a plant cell, a yeast cell, or a cell culture,
b) producing arachidonic acid by expression of the nucleic acid molecule according to claim 1 in the transgenic cell, and
c) obtaining arachidonic acid from the transgenic plant, the plant cell, the yeast cell, or the cell culture.

21. The method of claim 20, wherein the producing arachidonic acid by expression of the nucleic acid molecule further comprises the addition of dihomo-γ-linolenic acid.


SEQUENCE LISTING (6 sequences)

SEQ ID NO: 1 - DNA, 1514 bp, Phytophthora megasperma
  CDS (1)...(1434)
atggcccccatcgagactgtcaaagacgccaacgagggcctgcaccagcgcaagggcgccgctgccgcctccaaggacacgaccaccttcacgtggcaggacgtggccaagcacaacacggccaagagcgcctgggtcaccatccgcggcgtcgtctacgacgtgaccgaatgggccgaccgccaccccggaggccgcgagctcgtgctgctgcactcgggccgtgagtgcacggacacgttcgactcgtaccacccgttctccgaccgcgcggacaagatcctggccaagtacgccatcggcaagctcgtgggcggctccgagttccccacgtacaagcccgacacgggcttctacaaggagtgctgcgaccgcgtcaaccagtacttcaaggacaacaagctggacccgcgcagcccctactcgggtctgtggcgcatgatcctcgtggccatcgtcggcgctgtggcctacatgggcatgaaccagctgctgccgggcaacatctacgcgcactacgcgtggggcgcgctcttcggcgtgtgccaagcgctgccgctgctgcacgtcatgcacgacgcgtcgcacgctgccatcaccagcagccccacgggctggcggctcatcggccgcctcgcgatggactgggtggccggcgccaacatggtctcgtggctcaaccagcacgtggtgggccaccacatctacacgaacgtggcaggcgcggacccggatctgcccgtggacttcaagagcgacgtgcgccgcatcgtgtaccgccaggtgctgctgcccatttacaagtaccagcacctgtacctgccgccgctctacggcgtgctgggcctcaagttccgcgtgcaggacgtcttcgagacgttcgtcacgctgacgaatggcccgctgcgcgtgaacccgctctcggtgggcgactgggccgagatgatcctgtccaaggccttctgggtcttctaccgcatctacctcccgctggctgtgctccaggtggaccccgctcgcttctggggcgtcttcttcctcgccgagttctccacgggctggtacctggccttcaacttccaggtgagccacgtgtccacggcctgcgagtacccgggcggtgacgaggaagtgacgtccatcgacgacgagtgggccatctcgcaggtcaagtcgtcggtggactacggccacggctcgttcatcacgacgttcctcacgggcgcgctcaactaccaggtgacgcaccacctcttcccgggcgtctcgcagtaccattacccggccattgcgccgctcatcctcgacgtgtgccacaagtacaaggtcaagtacaacgtgctcccggacttcacggcggccatggccggccacttcgaccaccttgtcattatgggcaagatgggcaagcgcgtgaccatccacatgggctaagccagcagcggtcggttgaggacaaggccgtcttgcagctggactgcgactgctggtaagctttggctggataacgcggg

SEQ ID NO: 2 - protein, 477 aa, Phytophthora megasperma
MAPIETVKDANEGLHQRKGAAAASKDTTTFTWQDVAKHNTAKSAWVTIRGVVYDVTEWADRHPGGRELVLLHSGRECTDTFDSYHPFSDRADKILAKYAIGKLVGGSEFPTYKPDTGFYKECCDRVNQYFKDNKLDPRSPYSGLWRMILVAIVGAVAYMGMNQLLPGNIYAHYAWGALFGVCQALPLLHVMHDASHAAITSSPTGWRLIGRLAMDWVAGANMVSWLNQHVVGHHIYTNVAGADPDLPVDFKSDVRRIVYRQVLLPIYKYQHLYLPPLYGVLGLKFRVQDVFETFVTLTNGPLRVNPLSVGDWAEMILSKAFWVFYRIYLPLAVLQVDPARFWGVFFLAEFSTGWYLAFNFQVSHVSTACEYPGGDEEVTSIDDEWAISQVKSSVDYGHGSFITTFLTGALNYQVTHHLFPGVSQYHYPAIAPLILDVCHKYKVKYNVLPDFTAAMAGHFDHLVIMGKMGKRVTIHMG

SEQ ID NO: 3 - DNA, 27 bp, Artificial Sequence
  Oligonucleotide probe
gtggccaagcacaacacggccaagagc

SEQ ID NO: 4 - DNA, 30 bp, Artificial Sequence
  Oligonucleotide probe
accatccgcggcgtcgtctacgacgtgacc

SEQ ID NO: 5 - DNA, 31 bp, Artificial Sequence
  Oligonucleotide primer
ggatccatggcccccatcgagactgtcaaag

SEQ ID NO: 6 - DNA, 32 bp, Artificial Sequence
  Oligonucleotide primer
gcatgccccgcgttatccagccaaagcttacc